(19) United States
(12) Patent Application Publication
(10) Pub. No.: US 2006/0225195 A1
(43) Pub. Date: Oct. 12, 2006

(54) Antibacterial and antifungal material

(75) Inventor: Fred R. Scholer, East Windsor, NJ (US)

Correspondence Address: STROOCK & STROOCK & LAVAN LLP, 180 MAIDEN LANE, NEW YORK, NY, 10038, US

(73) Assignee: Hercules Chemical Company Incorporated

(21) Appl. No.: 11/104,775

(22) Filed: Apr. 12, 2005

(51) Int. Cl. E03D 3/00; C09D 5/16; A01N 47/10; A01N 55/02; A61K 31/27

(52) U.S. Cl. 4/300; 523/122; 514/184; 514/478; 514/504

(57) ABSTRACT

An anti-microbial package is provided that is suitable for use in products, for example, wax gaskets, in which the products themselves obtain enhanced anti-microbial characteristics. One particularly suitable product is a wax gasket ring for a toilet or urinal. The invention can be directed to an anti-microbial package preferably comprising one or more antifungal additives and one or more antibacterial additives, collectively “anti-microbial additives.” The package can also include a solvent suitable for dispersing them, preferably a solvent that is capable of substantially dissolving the one or more fungicide. Products containing the package and methods of producing those products are also provided.

### BACKGROUND OF THE INVENTION

The invention relates generally to antimicrobial compositions and methods and more particularly to bacteria and fungus inhibitor packages suitable for use in products that involve high temperature processing. Antimicrobial systems containing these packages are particularly suitable for use with wax seals, for example, wax seals for closet bowls and urinals, which are especially susceptible to bacteria and fungus growth.

Fungus and bacteria growth has continually been a health and environmental concern, especially for homeowners. Common fungi that plague houses include yeast and mold. Mold is particularly prevalent and easily spread. Whereas mold does not usually carry diseases, it can cause undesirable reaction in people who are sensitive to it. For example, mold can cause skin irritation, nasal stiffness, eye irritation, etc., and worse reaction can result from people who are allergic to mold.

Mold may or may not be visible to the naked eye, and resembles cobwebs or thread like masses. They can reproduce by releasing spores, which can be air, water or insect borne. The spores can adhere to a surface of an animate or inanimate object, especially objects comprising organic matter such as wood. Because mold cannot produce its own nutrients for survival, it needs a host from which they can absorb nutrients. Organic matter, such as wood or drywall can provide the nutrients that mold needs. Mold also requires a certain temperature and moisture in order for it to colonize and germinate, and certain conditions provide for more rapid germination than others. For example, warm, damp places are ideal for most molds. Common indoor molds include aureobasidium pullulans and aspergillus pullans, which can colonize in 2 to 3 days.

In addition to fungus, bacteria can be present in the home, particularly in the kitchen and bathrooms. Bacteria are microscopic, mostly unicellular, living organisms, which are often pathogens and can be responsible for non-hereditary diseases. Common methods of infection include contact, air, food, water and via insects.

A commonly known method of controlling fungi and bacteria is the use of chemicals or disinfectants and frequently cleaning the vulnerable areas. Certain vulnerable areas, however, are difficult to clean frequently because of their location. Moreover, it is difficult to produce products with built in antimicrobial activity. Antimicrobial coatings often wear off with time.

The Application of fungicide coating to various substrates are disclosed in U.S. Pat. Nos. 6,571,864, 5,882,731, 5,612,135, 4,742,093 and 4,662,403 and JP 2002 167551 and JP 2002 151602, which are all incorporated in their entirety herein by reference. However, application of the anti-microbial solution on some surfaces can be undesirable because it exposes the user to the anti-microbial agent. Additionally, the anti-microbial agent present on the surface of an object is likely not permanent, and may wear off after time.

In light of shortcomings of the conventional methods and applications known in the art, it is desirable to provide a package of anti-microbial agents that can be used to exhibit anti-microbial properties that can control fungal and bacterial growth, and products and methods of producing products comprising such package and exhibiting anti-microbial characteristics itself

### SUMMARY OF THE INVENTION

Generally speaking, in accordance with the invention, an anti-microbial package of anti-microbial additives suitable for use in products, for example, wax gaskets, in which the products themselves obtain enhanced anti-microbial characteristics. One particularly suitable product is a wax gasket ring for a toilet or urinal. The invention can be directed to an anti-microbial package preferably comprising one or more antifungal additives and one or more antibacterial additives, collectively “anti-microbial additives.” The package can also include a solvent suitable for dispersing them, preferably a solvent that is capable of substantially dissolving the one or more fungicide. Products containing the package and methods of producing those products are also provided.

The anti-microbial package can comprise any or a combination of one or more of: families of phenols, sodium phenate/phenols, and chlorinated phenols, chlorinated melamines, active quaternary ammonium chloride germicides, such as dialkylarylammonium salts, nonpathogenic spore based bacteria, iodines and iodophores, substituted hydantoins, thiadiazine-thiones, substituted butyl carbamates, substituted triazine-diammines, benisothiazolin-3-ones and/or brominated nitrilopropionamides. Examples of preferred anti-microbial additives include, but are not limited to, oxybisphenoxyarsine (OBPA), 2-n-octyl-4-isothiazolin-3-one (OIT), 3-iodo-2-propyl butyl carbamate (IPBC), zinc 2-pyridinethiol-n-oxide (ZNP), triclosan and organotin compounds. It is also advantageous to include elemental silver or anti-microbial silver ion containing materials. The most preferred anti-microbial package includes a combination of ZNP and at least one of IPBC, OBPA, OIT and/or natural antimicrobial sources, such as glucosinolates, for example, allylisothiocyanate (AIT).

The additives are preferably included in about a 2:1 to 1:4 ratio, more preferably 1:1 to 1:3, most preferably about a 1:2 ratio. The preferred level of the anti-microbial additives is 100-1000 ppm in the object (e.g., the wax gasket), more preferably 300-800 ppm. In one preferred embodiment of the invention, the

According to a preferred use of an embodiment of the invention, the anti-microbial additives are added to a substance, such as wax, during manufacture. Therefore, preferred additive embodiments of the invention are stable in temperatures employed in manufacturing the wax gaskets, for example, 100-180° F., and maintain their anti-microbial properties after manufacture. Most preferably, the anti-microbial additives maintain over 80% and up to 100% of their anti-microbial properties after manufacture.

A wax gasket comprising a package of anti-microbial additives according to an embodiment of the invention can prevent the growth of mold and bacteria under and on the surface of the wax gasket, and further comprises a protective zone of inhibition preventing the encroachment of bacteria, mold and other fungi into the area surrounding the wax gasket. Preferred zones of inhibition are at least 1.5 inches in diameter, more preferably at least 2.0 inches in diameter.

Additionally, a package of anti-microbial additives formed in accordance with an embodiment of the invention will not significantly alter the manufacturing process of wax gaskets and will not substantially raise the cost of manufacture or the performance characteristics of the gasket.

Accordingly, it is an objective of the invention to provide a package of improved anti-microbial additives and methods and products employing those additives.

Another object of the invention is to provide an improved wax gasket and method of manufacture.

Still other objects and advantages of the invention will in part be obvious and will in part be apparent from the specification and drawings.

The invention accordingly comprises the several steps and the relation of one or more of such steps with respect to each of the others, and the article of construction and composition embodying combinations of elements which result from such steps, all as exemplified in the following detailed disclosure, and the scope of the invention will be indicated in the claims.

### BRIEF DESCRIPTION OF THE DRAWINGS

For a fuller understanding of the invention, reference is had to the following description, taken in connection with the accompanying drawings, in which:

FIG. 1 is a cut-away side view of a toilet having an object constructed in accordance with an embodiment of the present invention.

FIG. 2A is a schematic diagram of a dish containing an object constructed in accordance with an embodiment of the present invention.

FIG. 2B is a schematic diagram of a dish containing an object constructed in accordance with an embodiment of the present invention.

FIG. 2C is a schematic diagram of a dish containing an object constructed in accordance with an embodiment of the present invention.

FIG. 2D is a schematic diagram of a dish containing an object constructed in accordance with an embodiment of the present invention.

FIG. 3A is a schematic diagram of a dish containing a prior art object.

FIG. 3B is a schematic diagram of a dish containing an object constructed in accordance with an embodiment of the present invention.

### DESCRIPTION OF THE PREFERRED EMBODIMENTS

An antimicrobial system in accordance with the invention can comprise an antimicrobial package comprising antimicrobial additives, including, but not limited to, fungicides and/or bactericides and one or more solvents. Toilets, closet bowls, urinals and the like (hereinafter collectively “toilets”) drain into pipes. A wax gasket is commonly used as a seal at the junction with the pipe. Wax can serve as a nutrient source for mold and other microbes. It has been determined that the location of the gasket, shielded from the light, in a hard to clean location of a bathroom make it a particularly suitable object to be made in accordance with the invention by the addition of antimicrobials. Other suitable objects include plastic pipes, fittings, gaskets, food preparation surfaces and other objects. An anti-microbial agent or solution as discussed herein includes a chemical and/or system that displays antagonistic behavior against both fungi and bacteria.

Commercially available chemicals and disinfectants are considered inadequate remedies for removing and/or preventing mold and bacteria in susceptible objects and locations that are difficult to reach. Dipping or spraying the wax ring with an appropriate solution of an anti-microbial agent during the molding process is also considered inadequate. Alternatively, the wax ring can be sprayed with antimicrobial materials by the installer.

In accordance with an embodiment of the invention, the antimicrobial package comprises at least one, preferably a combination of two or more antimicrobial additives. Thus, both antibacterial and antifungal activity can be obtained. Preferably, the antimicrobial additives are stable in temperatures employed in the manufacture of the products in which they are to be incorporated. For example, for inclusion in wax ring gaskets, they should be stable between about 100° to 180° F. The term “stable” thus means the additives remain chemically intact and do not lose their efficacy as an antibacterial or antifungal additive.

The antimicrobial package should be safe to use in the manufacturing environment and provides a substantially no growth (microbistatic) environment under and on the surface of the object during use. Furthermore, a zone of inhibition, an area surrounding the object in which substantially no fungus grow, is preferably created, surrounding the object. Typically, bacteria will also not grow in this region, but the dimensions could be different. For wax rings as toilet gaskets, suitable zones of inhibition include areas preferably having a diameter of at least about 1.5 inches, more preferably at least about 2.0 inches. The absence of fungal or bacterial growth can be established by testing the object, e.g., the wax rings, under conditions described by AATCC Method 147, more specifically, the Parallel Streak Method. This test determines antimicrobial activity of diffusible anti-microbial agents from a treated substrate, demonstrating bacteriostatic activity by the diffusion of the antimicrobial agent through the surface inoculated with test organisms. ASTM D3273 can also be used. This test evaluates the relative resistance of films to surface mold fungi and mildew growth in a severe interior environment. Another test can be performed under the conditions described by the National Committee on clinical Laboratory Standards (NCCLS).

By way of nonlimiting example, using a wax ring constructed in accordance with an embodiment of the invention can result in a clear zone of no growth on the surface, under or surrounding the sample. The formation of the zone of inhibition can be attributed to the diffusion of the antimicrobial additives out of the sample and into the surrounding area. The antimicrobial additives can migrate to the surface of the wax and inhibit the growth or kill the organisms including fungi and bacteria. The size of the zone of inhibition can depend on the diffusion rate, the effectiveness of the antimicrobial additives and the growth rate of the organism.

In accordance with a preferred embodiment of the invention, the antimicrobial additives include one or more antifungal additives and one or more antibacterial additives. Preferred antifungal additives can include, but are not limited to, families of phenols, sodium phenate/phenols, chlorinated phenols, chlorinated melamines, iodines and iodophores, substituted butyl carbamates, substituted triazine-diammines, benisothiazolin-3-ones, brominated nitrilopropionamides and organotin compounds. The most preferred antifungal additives include oxybisphenoxyarsine (OBPA) and 3-iodo-2-propyl butyl carbamate (IPBC), of which IPBC is most preferred.

Preferred antibacterial additives can include, but are not limited to, families of phenols, sodium phenate/phenols, chlorinated phenols, chlorinated melamines, active quaternary ammonium chloride germicides, such as dialkylarylammonium salts, nonpathogenic spore based bacteria, iodines and iodophores, substituted hydantoins, brominated nitrilopropionamides and organotin compounds. The more preferred antibacterial additives include zinc 2-pyridinethiol-n-oxide (ZNP) and triclosan, of which ZNP is most preferred.

According to a preferred embodiment of the invention, the antimicrobial additives comprise IPBC and ZNP. The IPBC can comprise a commercially available IPBC sold under the trademark Polyphase® AF-1, manufactured and sold by Troy Corporation of Florham Park, N.J., and the ZNP can comprise a commercially available ZNP sold under the trademark Intercide ZNP Powder, manufactured and sold by Ackros Chemicals America Inc.

In accordance with a preferred embodiment of the invention, the effective level of antimicrobials additives, which produces a zone of inhibition, comprises approximately 0.01-0.08% (100-800 ppm) by weight. According to a preferred embodiment of the invention, an antifungal additive comprises about 0.01-0.10% and an antibacterial additive comprises about 0.005 to 0.05% by weight. Among the four categories of the efficacy of additives: no growth, zone of inhibition; no growth, no zone of inhibition; growth fails, no zone; and stain fails, the preferred embodiment falls within the “no growth, zone of inhibition” category.

It is preferred for the antimicrobial additives to be dissolved or dispersed in a solvent prior to being added to the wax. As used herein, “wax” will include all fully refined waxes, which are derivatives of petroleum waxes comprising a blend of micro and macro-crystalline waxes, as well as synthetic waxes derived fatty acids of amides or non-fossil waxes based on animal or vegetable waxes. Micro and macro-crystalline waxes can comprise straight-chain alkanes from C-18 to C-45. Typically, macro-waxes can be straight chain linear alkanes and micro-waxes can have branched chains or isoalkane content as well as a higher naphthenic content.

Preferably, the antimicrobial additives are dissolved or dispersed in common solvents that are compatible with the wax and preferably significantly dissolves or disperses the fungicide. Suitable solvents include, but are not limited to, ketones, pyrrilidones, glycols, glycol ethers, ethoxylated alkyl phenols, alkoxylated linear alcohols, alkanolamines, aromatic solvents, hydrocarbons and cyclic ethers. Examples of preferred solvents include, but are not limited to, dimethyl sulfoxide, N-methylpyrrlidone, cyclohexanone, dioxalane, propylene glycol, glycol ether DB, MEK, hexane, o-xylene, dodecane, dimethoxyethane, toluene and mineral spirits.

In accordance with an embodiment of the invention, an antimicrobial additive, preferably a fungicide, more preferably IPBC, is first dissolved in a solvent. A second antimicrobial additive, preferably a bactericide, most preferably ZnP, is then dispersed in the mixture. Alternatively, a solid anti-microbial additive can be dispersed in a liquid antimicrobial additive. For example, zinc pyrithiones can be dispersed in an IPBC, such as Troy Polyphase® AF-1, which is a manufacturer made IPBC dissolved in solvents.

Preferably, a substance, such as wax, comprises an antimicrobial package in accordance with an embodiment of the invention dispersed therein. For example, the antimicrobial package comprising antimicrobial additives dissolved or dispersed in a solvent can be added to hot wax, preferably in a holding tank, for recycling or mixing with the hot wax. The hot wax can be held in the holding tank at a suitable temperature. For example, a high grade petroleum wax can be held at temperatures exceeding 150° F. The antimicrobial package can then be added into the holding tank, and subsequently mixed with the hot wax. After mixing, the hot wax can be poured into molds, in which it can be cooled, and then removed. This will provide an object with longer lasting antimicrobial properties than an object that is merely coated with antimicrobial materials on its surface.

Preferably, an antimicrobial package in accordance with the invention is dispersible throughout a hot wax medium and is stable at high temperatures that are used in producing wax products, such as wax rings. The antimicrobial package preferably maintains its antifungal and antibacterial properties throughout the production of the wax ring, as well as after production is completed, thereby exhibiting the antifungal and antibacterial properties when the wax ring is in use. Preferably, the antimicrobial package prevents growth of fungus and bacteria on the surface of the wax and more preferably, exhibits a zone of inhibition, an area surrounding the wax in which no fungus or bacteria can grow.

In addition to the antimicrobial package's ability to maintain its antifungal and antibacterial properties and performance during and after production of the wax rings, it is preferred for the antimicrobial package not to have a detrimental effect on the properties of performance of the ring itself.

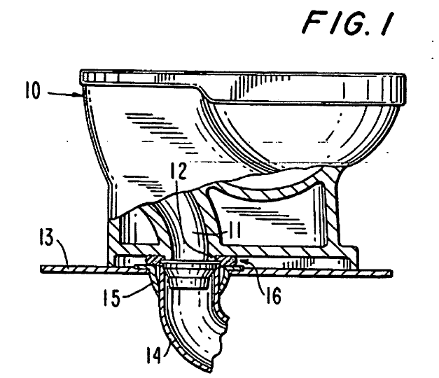

As shown in FIG. 1, a wax ring 16 constructed in accordance with an embodiment of the invention can be positioned between a discharge outlet 11, preferably having a horn 12, of a toilet 10 and a flange 15 of a waste pipe 14 extending through a floor 13. Wax ring 16 can weigh about 120-140 g and can be in the shape of a ring or doughnut having an inner and outer diameter of about 18 cm and 20 cm, respectively. Wax ring 16 can be about 1.5 cm high.

Embodiments of the invention will be discussed more specifically with reference to the following examples, which are presented for purposes of illustration only and are not intended to be construed as limiting.

Samples of wax comprising antimicrobial packages described below in accordance with embodiments of the invention were tested with Aspergillus pullans (ATCC #6275), Aureobasidium pullulans (ATCC #9348) and Chaetomium globosum (ATCC #). Samples were tested for fungal/mildew resistance in accordance with ASTM D3273, which evaluates the relative resistance of films to surface mold fungi, mildew growth in a severe environment. The samples were tested for a period of 4 weeks at 90° F. and a relative humidity of 95% in an ASTM environmental Mold Chamber. The samples were further tested with the same organisms using the Parallel Streak Method as described in AATCC Test Method 147. Both tests demonstrated a resistance to these organisms as demonstrated by the lack of growth on the surface of the samples as well as by a zone of inhibition created surrounding the test area. A zone of inhibition study was performed in accordance with NCCLS in order to measure the effect of concentration changes on the bacteriostatic behavior of the samples.

The sample wax rings were also tested against control wax rings without antimicrobial additives. The test employed was according to ASTM D-938, at 77° F. and at 100° F. by Cone Penetration, using a device having a weighted needle at its tip, which is allowed to penetrate into the wax. The depth of the penetration helps measure the softness of the wax. The properties measured in the test include Congealing Point, which demonstrates the temperature at which a liquid wax melt first begins to show signs of solid formation upon cooling, and a Flexural Strength Bend test at room temperature, which measures the resistance to breakage upon bending. The Flexural Strength Bend test is not an ASTM test but was developed by Hercules Chemical Company, Inc. of Passaic, N.J. More specifically, according to the Flexural Strength Bend test, a donut ring of wax is bent in half, whereupon the ring preferably cracks and separates. If the point of breakage appears to resemble taffy or soft candy, the ring can be deemed to have insufficient strength. If the point of breakage comprises thread or fiber type structure and the break is relatively clean, it can indicate that the wax ring comprises internal microcrystalline structure and that the wax ring can withstand the stress of the weight of a toilet. According to the tests performed, the sample wax rings demonstrated the same physical properties tested as the control wax rings.

The following examples are provided for illustration and to provide a better description of the invention. They are not intended to be limiting.

### EXAMPLE 1

Composition:

| | |
|---|---|
| 1. Troy Polyphase ® AF-1 | 0.1 |
| 2. Petroleum Based Wax | 99.9 |
| | 100 |

Method: The Troy Polyphase® AF-1 was stirred into hot wax maintained at 150° F. 120 grams of the wax mixture was poured into an aluminum mold in the shape of a donut having an inside diameter (ID) of approximately 4 inches and an outside diameter (OD) of approximately 6 inches. The wax ring was cooled and then removed from the mold and 4 random slices were taken from the ring and each slice was placed in the center of an agar dish.

A control wax ring was made containing 100% of the same petroleum based wax with no additives. Four random slices were taken from the control wax ring, and each slice was placed in the center of an agar dish. The dish was inoculated with aspergillus pullans and the samples and controls were tested according to NCCLS.

Result: Samples containing the antimicrobial agent in accordance with the invention exhibited no growth and had an established zone of inhibition having a diameter of at least 1.5 inches. The control, on the other hand, exhibited growth of fungus up to and over the wax sample at the center of the Petri dish.

### EXAMPLE 2

| | |
|---|---|
| 1. Troy Polyphase ® AF-1 | 0.04 |
| 2. Ackros Intercide ZNP | 0.02 |
| 2. Petroleum Based Wax | 99.94 |
| | 100.00 |

Method: The Troy Polyphase® AF-1 and Ackros Intercide ZNP were stirred into hot wax maintained at 150° F. 120 grams of the wax mixture were poured into an aluminum mold in the shape of a donut having an ID of approximately 4 inches and an OD of approximately 6 inches. The wax ring was cooled and then removed from the mold and 4 random slices were taken from the ring. A control wax ring was made by forming a ring of 100% wax with no additives. Four random slices were taken from the control ring.

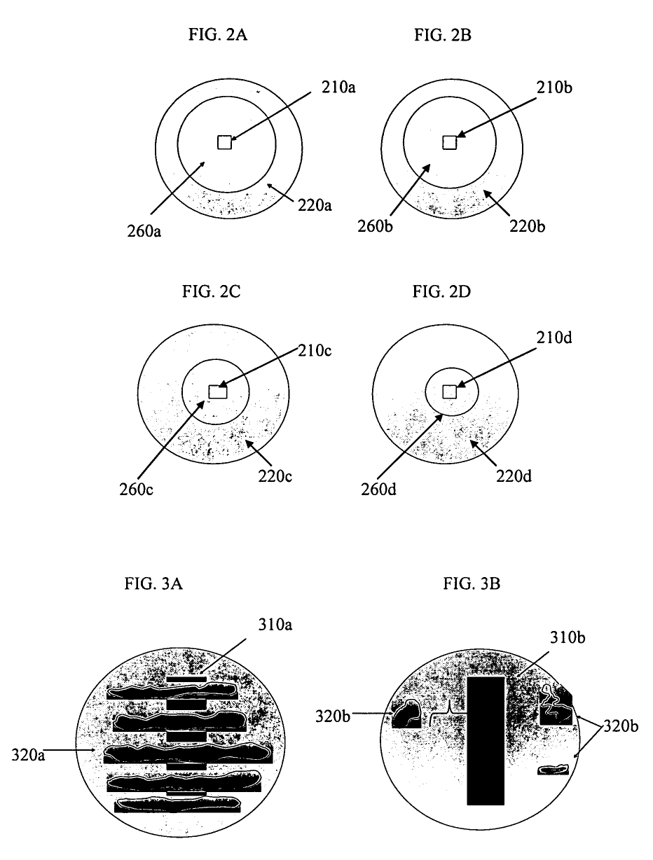

In accordance with the techniques described in NCCLS and AATCC Test Method 147, four wax ring samples were prepared containing relative amounts of the antimicrobial agent in ratios of 1:0.5:0.25:0.125, as illustrated in FIGS. 2A, 2B, 2C and 2D, respectively. 5 cubic mm wax samples 210a, 210b, 210c and 210d were cut from each of the four different rings. Each sample was placed at the center of an agar plate 250, which was then inoculated with Aspergillus pullans. As shown, a large zone of inhibition 260a and relatively smaller zones of inhibition 260b, 260c and 260d were observed. Although the zones of inhibition 260a, 260b, 260c and 260d decrease in size relative to concentration, a dominant zone appears at all selected levels.

The samples and controls were also tested according to the Parallel Streak Method as shown in FIGS. 3A-B, which is a relatively quick and easily executed qualitative method to determine antimicrobial activity of diffusible anti-microbial agents from a treated substrate. This test demonstrates bacteriostatic activity by the diffusion of the antimicrobial agent through the agar, in which the agar surface is inoculated with the test organisms by making five streaks approximately 60 mm in length spaced 10 mm apart covering the central area of the Petri dish. A specimen is transversely pressed across the five inoculum streaks to ensure intimate contact with the agar surface. FIG. 3A shows a wax control sample 310a that has not been treated with an antimicrobial package, and FIG. 3B shows a wax test sample 310b that was treated with an antimicrobial package in accordance with the invention, both of which were placed in a Petri dish and inoculated with aureobasidium pullulans using the Parallel Streak Method.

Results:

Samples comprising the antimicrobial package in accordance with the invention demonstrated a zone of inhibition 260a, 260b, 260c and 260d, and only areas 220a, 220b, 220c and 220d outside zone of inhibition 260a, 260b, 260c and 260d were infected. As shown in FIGS. 2A-D, each successive lower concentration of the antimicrobial package results in a smaller zone of inhibition 260a, 260b, 260c and 260d surrounding wax sample 210a, 210b, 210c and 210d. However, it is notable that a zone of inhibition is present for each concentration.

As shown in FIG. 3B, the inoculum streaks along side and under wax test sample 310b, which was treated with an antimicrobial package in accordance with an embodiment of the invention, displayed no growth on top of, under or within a zone of inhibition 360b created around the samples. Colonies of aureobasidium pullulans 320b were present only in areas remote from wax test sample 310b and outside zone of inhibition 360b. In contrast, as shown in FIG. 3A, colonies of aureobasidium pullulans 320a were present freely throughout the Petri dish, including on top of and under wax control sample 310a.

It will thus be seen that the objects set forth above, among those made apparent from the preceding description, are efficiently attained, and, since certain changes may be made in carrying out the above method and in the compositions set forth without departing from the spirit and scope of the invention, it is intended that all matter contained in the above description and shown in the accompanying drawings shall be interpreted as illustrative and not in a limiting sense. For example, whereas petroleum wax is described in the examples, it is to be understood that the object can comprise any composition suitable for high temperature processing and manufacture.

It is also to be understood that the following claims are intended to cover all of the generic and specific features of the invention herein described and all statements of the scope of the invention, which, as a matter of language, might be said to fall therebetween.

Particularly it is to be understood that in said claims, ingredients or compounds recited in the singular are intended to include compatible mixtures of such ingredients wherever the sense permits.

1. A toilet system including a toilet connected to a drain pipe with a wax gasket therebetween, the wax gasket comprising:
a wax-like material with an anti-microbial additive comprising at least one anti-bacterial additive and at least one anti-fungal additive combined therewith and providing a zone of inhibition at least about 1.5 inches from the wax gasket, as established under conditions described by AATCC Method 147.

2. The toilet system of claim 1, wherein the anti-microbial additive comprises at least one member selected from the group consisting of phenols, chlorinated melamines, active quaternary ammonium chloride germicides, iodines, iodophores, substituted hydantoins, substituted butyl carbamates, substituted triazine-diammines, benisothiazolin-3-ones, brominated nitrilopropionamides and/or organotin compounds.

3. The toilet system of claim 1, wherein the wax gasket comprises at least one of zinc 2-pyridinethiol-n-oxide, triclosan, oxybisphenoxyarsine and/or 3-iodo-2-propyl butyl carbamate.

4. The toilet system of claim 1, wherein the wax gasket comprises about 200 to 1000 ppm of anti-microbial additive.

5. The toilet system of claim 1, wherein the wax gasket comprises 0.01 to 0.10% by weight of the anti-fungal additive and 0.005 to 0.05% by weight of the anti-bacterial additive.

6. A composition resulting from the room temperature combination of components heated to a temperature sufficiently high to soften the components to permit them to be mixed together, comprising:
a wax-like material;
an antimicrobial additive mixed into the material, the antimicrobial additive comprising an anti-bacterially effective amount of at least one anti-bacterial additive and an anti-fungally effective amount of at least one anti-fungal additive;
wherein the additives maintain at least 80% of their antimicrobial efficacy after the combination is cooled to room temperature.

7. The composition of claim 6, wherein the additives have been heated to a temperature of over 125° F. for over about 10 minutes.

8. The composition of claim 6, wherein the additives have been heated to a temperature of over 125° F. for over about one hour.

9. The composition of claim 6, wherein the antimicrobial additive comprises phenols.

10. The composition of claim 6, wherein the antimicrobial additive comprises at least one of chlorinated melamines, active quaternary ammonium chloride germicides, substituted hydantoins, brominated nitrilopropionamides and/or organotin compounds.

11. The composition of claim 10, wherein the active quarternary ammonium chloride germicides comprise at least one of dialkylarylammonium salts, nonpathogenic spore based bacteria, iodines and/or iodophores.

12. The composition of claim 6, wherein the antimicrobial additive comprises at least one of zinc 2-pyridinethiol-n-oxide and/or triclosan.

13. The composition of claim 6, wherein the antimicrobial additive comprises at least one of chlorinated melamines, iodines and/or iodophores.

14. The composition of claim 6, wherein the antimicrobial additive comprises at least one of substituted butyl carbamates, substituted triazine-diammines, benisothiazolin-3-ones, brominated nitrilopropionamides and/or organotin compounds.

15. The composition of claim 6, wherein the antimicrobial additive comprises at least one of oxybisphenoxyarsine and/or 3-iodo-2-propyl butyl carbamate.

16. The composition of claim 6, comprising a solvent that substantially dissolves or disperses the anti-fungal additive.

17. The composition of claim 6, comprising a solvent that substantially dissolves or disperses the anti-bacterial additive.

18. The composition of claim 6, wherein the solvent comprises at least one of ketones, pyrrilidones, glycols, glycol ethers, ethoxylated alkyl phenols, alkoxylated linear alcohols, alkanolamines, aromatic solvents, hydrocarbons and/or cyclic ethers.

19. The composition of claim 6, wherein the solvent comprises at least one of dimethyl sulfoxide, N-methylpyrrlidone, cyclohexanone, dioxalane, propylene glycol, glycol ether DB, MEK, hexane, o-xylene, dodecane, dimethoxyethane, toluene and/or mineral spirits.

20. The composition of claim 6, comprising an effective amount of at least one anti-bacterial additive and at least one anti-fungal additive to create a zone of inhibition of over 1.5 inches, as established under conditions described by AATCC Method 147.

21. The composition of claim 20, wherein the zone of inhibition comprises a diameter of at least about 2 inches.

22. The composition of claim 6, wherein the ratio of anti-bacterial additive to anti-fungal additive is between about 2:1 to about 1:4.

23. The composition of claim 6, wherein the ratio of anti-bacterial additive to anti-fungal additive is between about 1:1 to about 1:3.

24. The composition of claim 6, comprising about 200 to 1000 ppm antimicrobial additive in the composition.

25. The composition of claim 6, comprising about 300 to 800 ppm antimicrobial additive in the composition.

26. The composition of claim 6, wherein the wax-like material consists essentially one or more waxes selected from the group consisting of petroleum waxes, petroleum wax derivatives, micro and/or macro-crystalline waxes, synthetic waxes derived fatty acids of amides, non-fossil waxes based on animal or vegetable waxes, and micro-crytsalline waxes and/or macro-crystalline waxes comprising straight-chain alkanes from C-18 to C-45.

27. The composition of claim 6, comprising 0.01 to 0.10% by weight of the anti-fungal additive, 0.005 to 0.05% by weight of the anti-bacterial additive.

28. The composition of claim 6, comprising 3-iodo-2-propyl butyl carbamate and zinc 2-pyridinethiol-n-oxide.

29. The composition of claim 6, wherein a zone of inhibition can be maintained for at least four weeks at 90° F. and a relative humidity of 95% in a mold chamber.

30. A method of making a wax gasket structure, comprising:
providing a formulation comprising at least one antimicrobial additive and a solvent;
providing melted wax;
mixing the formulation into the hot wax;
pouring the hot wax into one or more molds; and
cooling the hot wax;
wherein the molded wax provides a zone of inhibition of at least 1.5 inches.

31. The method of claim 30, wherein the molded wax comprises 0.01 to 0.10% by weight of an anti-fungal additive and 0.005 to 0.05% by weight of an anti-bacterial additive.

32. The method of claim 30, wherein the antimicrobial additive comprises at least one of phenols, chlorinated melamines, active quaternary ammonium chloride germicides, substituted hydantoins, brominated nitrilopropionamides and/or organotin compounds.

33. The method of claim 30, wherein the antimicrobial additive comprises zinc 2-pyridinethiol-n-oxide and/or triclosan.

34. The method of claim 30, wherein the antimicrobial additive comprises at least one of families of phenols, sodium phenate/phenols, chlorinated phenols, chlorinated melamines, iodines, iodophores, substituted butyl carbamates, substituted triazine-diammines, benisothiazolin-3-ones, brominated nitrilopropionamides and/or organotin compounds.

35. The method of claim 30, wherein the antimicrobial additive comprises at least one of oxybisphenoxyarsine and/or 3-iodo-2-propyl butyl carbamate.

36. The method of claim 30, wherein the formulation comprises 3-iodo-2-propyl butyl carbamate and zinc 2-pyridinethiol-n-oxide.
